(19) United States
(12) Patent Application Publication
(10) Pub. No.: US 2011/0034524 A1
(43) Pub. Date: Feb. 10, 2011

(54) HEMI-FUMARATE SALT OF A 1,3,4-THIADIAZOLYL DERIVATIVE AS MODULATOR OF THE SPHINGOSINE 1-PHOSPHATE RECEPTOR

(75) Inventors: Michael Simon Anson, Stevenage (GB); Caroline Jane Day, Stevenage (GB); Jonathan Paul Graham, Stevenage (GB); Leanda Jane Kindon, Stevenage (GB); Grahame Robert Woollam, Stevenage (GB)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: Glaxo Group Limited, Greenford (GB)

(21) Appl. No.: 12/866,156

(22) PCT Filed: Feb. 3, 2009
(86) PCT No.: PCT/EP2009/051175

§ 371 Date: Aug. 4, 2010

(30) Foreign Application Priority Data
Feb. 6, 2008 (GB) 0802227.9

(51) Int. Cl. A61K 31/433; C07D 285/13; A61P 29/00; A61P 37/06; A61P 11/06; A61P 19/02; A61P 35/00; A61P 17/06; A61P 35/02; A61P 31/12; A61P 3/10

(52) U.S. Cl. 514/363; 548/136

(57) ABSTRACT

The invention relates to the compound (2S)-2-amino-2-{5-[4-(octyloxy)-3-(trifluoromethyl)phenyl]-1,3,4-thiadiazol-2-yl}-1-propanol hemi-fumarate, processes for its preparation, pharmaceutical compositions containing them and its use in the treatment of conditions or disorders which are mediated via the S1P1 receptor.

The present invention relates to a novel thiadiazole derivative having pharmacological activity, processes for its preparation, pharmaceutical compositions containing it and its use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandala et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 EurJ Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe compounds useful as S1P1 agonists: WO 04/024673, WO 04/096752, WO 02/18395, WO 03/061567, WO 02/064616, WO 04/010949, U.S. Pat. No. 7,064,217 and WO 05/041899.

International patent application PCT/US2007/017282 (WO 08/016,692) describes thiadiazole derivatives as agonists of the S1P1 receptor, including (2S)-2-amino-2-{5-[4-(octyloxy)-3-(trifluoromethyl)phenyl]-1,3,4-thiadiazol-2-yl}-1-propanol and pharmaceutically acceptable salts thereof.

A novel salt of (2S)-2-amino-2-{5-[4-(octyloxy)-3-(trifluoromethyl)phenyl]-1,3,4-thiadiazol-2-yl}-1-propanol has now been found which may be of use in the treatment of disorders mediated via the S1P1 receptor.

The present invention provides (2S)-2-amino-2-{5-[4-(octyloxy)-3-(trifluoromethyl)phenyl]-1,3,4-thiadiazol-2-yl}-1-propanol hemi-fumarate.

One advantage of the hemi-fumarate salt is its readiness to establish a stable crystalline form. This invention includes within its scope stoichiometric hydrates or solvates as well as compounds containing variable amounts of water and/or solvent.

Included within the scope of the invention are all solvates, hydrates, polymorphs, prodrugs, of the compound of the invention.

In a further aspect, this invention provides processes for the preparation of the compound of the invention, which may be prepared, by treating (2S)-2-amino-2-{5-[4-(octyloxy)-3-(trifluoromethyl)phenyl]-1,3,4-thiadiazol-2-yl}-1-propanol (prepared by any conventional methods, including those described in PCT/US2007/017282) with fumaric acid.

In another aspect, this invention provides the following process for the preparation of the compound of the invention:

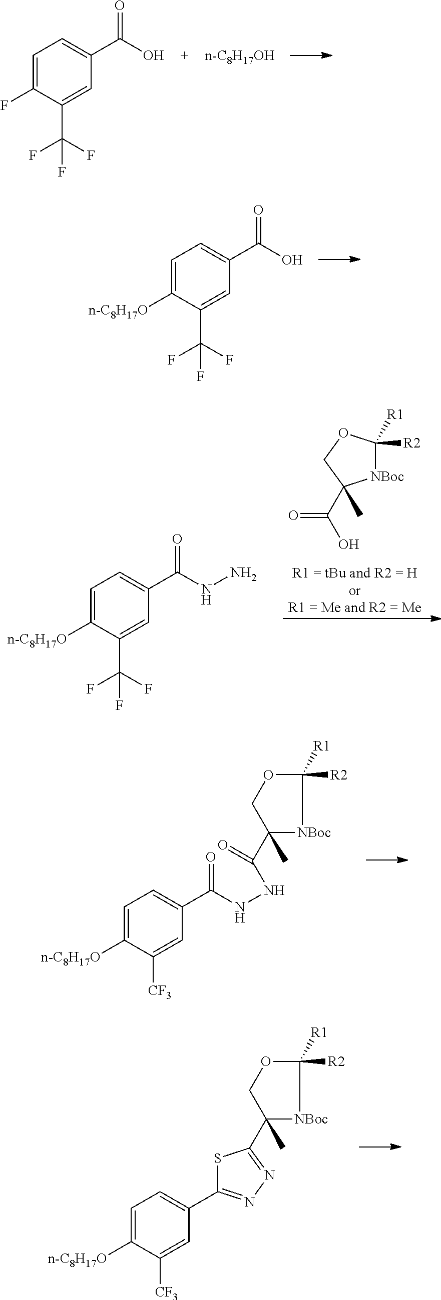

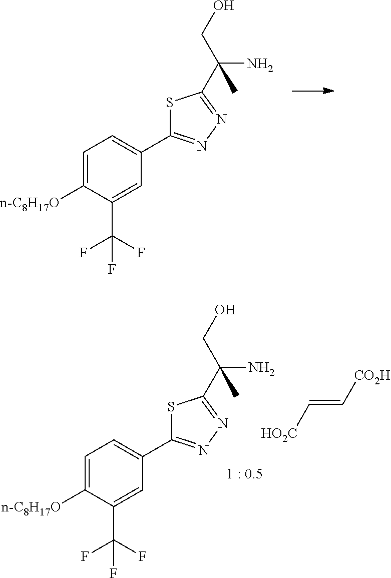

The potencies and efficacies of the compound of this invention for the S1P1 receptor can be determined by GTPγS assay performed on the human cloned receptor as described herein. The compound of the invention has agonist activity at the S1P1 receptor, using functional assays described herein.

The compound of the invention is therefore of use in the treatment of conditions or disorders which are mediated via the S1P1 receptor. In particular the compound of the invention is of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes (herein after referred to as the “Disorders of the Invention”).

The compound of the invention is therefore of use in the treatment of lupus erythematosis.

The compound of the invention is therefore of use in the treatment of psoriasis.

The compound of the invention is therefore of use in the treatment of multiple sclerosis.

It is to be understood that “treatment” as used herein includes prophylaxis as well as alleviation of established symptoms.

Thus, the invention also provides the compound of the invention for use as a therapeutic substance, in particular in the treatment of the conditions or disorders mediated via the S1P1 receptor. In particular the invention provides the compound of the invention for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

The compound of the invention is therefore of use as a therapeutic substance in the treatment of lupus erythematosis.

The compound of the invention is therefore are of use as a therapeutic substance in the treatment of psoriasis.

The compound of the invention is therefore of use as a therapeutic substance in the treatment of multiple sclerosis.

In another aspect, the invention provides for the use of the compound of the invention in the manufacture of a medicament for use in the treatment of the conditions or disorders mediated via the S1P1 receptor.

The compound of the invention is of use in the manufacture of a medicament for use in the treatment of lupus erythematosis.

The compound of the invention is of use in the manufacture of a medicament for use in the treatment of psoriasis.

The compound of the invention is of use in the manufacture of a medicament for use in the treatment of multiple sclerosis.

The invention further provides a method of treatment of conditions or disorders in mammals including humans which can be mediated via the S1P1 receptor, which comprises administering to the sufferer a therapeutically safe and effective amount of the compound of the invention.

The invention provides a method of treatment of lupus erythematosis, which comprises administering to the sufferer a therapeutically safe and effective amount of a compound of the invention.

The invention provides a method of treatment of psoriasis, which comprises administering to the sufferer a therapeutically safe and effective amount of a compound of the invention.

The invention provides a method of treatment of multiple sclerosis, which comprises administering to the sufferer a therapeutically safe and effective amount of a compound of the invention.

In order to use the compound of the invention thereof in therapy, it will normally be formulated into a pharmaceutical composition in accordance with standard pharmaceutical practice. The present invention also provides a pharmaceutical composition, which comprises the compound of the invention, and a pharmaceutically acceptable carrier or excipient.

In a further aspect, the present invention provides a process for preparing a pharmaceutical composition, the process comprising mixing the compound of the invention and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compound of the invention may also be formulated in rectal compositions such as suppositories or retention enemas, e.g. containing conventional suppository bases such as cocoa butter or other glycerides.

The compound of the invention may also be formulated as depot preparations. Such long acting formulations may be administered by implantation (for example subcutaneously or intramuscularly) or by intramuscular injection. Thus, for example, the compound of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compound of the invention, may be formulated as solutions for administration via a suitable metered or unitary dose device or alternatively as a powder mix with a suitable carrier for administration using a suitable delivery device. Thus the compound of the invention may be formulated for oral, buccal, parenteral, topical (including ophthalmic and nasal), depot or rectal administration or in a form suitable for administration by inhalation or insufflation (either through the mouth or nose).

The compound of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

The compound of the invention may be used in combination preparations. For example, the compound of the invention may be used in combination with cyclosporin A, methotrexate, steroids, rapamycin, proinflammatory cytokine inhibitors, immunomodulators including biologicals or other therapeutically active compounds.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of the compound of the invention.

### Abbreviations:

g—grams
mg—milligrams
kg—kilograms
ml—millilitres
L—litres
RT—room temperature
° C.—degrees Celsius
sat.—saturated
NaHMDS—sodium bis(trimethyldisilyl)amide
TBME—tert-butyldimethyl ether
HCl—hydrochloric acid
MeTHF—2-methyltetrahydrofuran
THF—tetrahydrofuran
NaHCO₃—sodium bicarbonate
CDl—1,1′carbonyldiimidazole
conc.—concentrated

### General Chemistry Section

The intermediates for the preparation of the examples may not necessarily have been prepared from the specific batch described.

### Description 1

### 4-(octyloxy)-3-(trifluoromethyl)benzoic acid (D1)

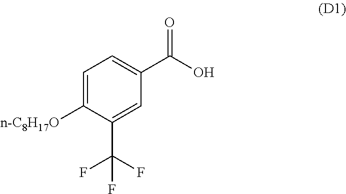

A 1.5M solution of NaHMDS in THF (9 vol; 2.8 eq) is diluted with THF (4.5 vol) and n-octanol (1.52 vol; 2 eq) is charged and heated to 55-60° C. A solution of 4-fluoro-3-(trifluoromethyl)benzoic acid (1 wt., 1 eq) in THF (2 vol) is charged, washing through with further THF (1 vol). The reaction mixture is stirred at 57±3° C. until deemed complete by HPLC (ca. 24 h, <4% area 4-fluoro-3-(trifluoromethyl)benzoic acid remaining). The reaction is then cooled to 20-25° C. and solvent swapped into water (10 vol) by vacuum distillation (remove ca. 15 vol distillate) to afford a tan slurry which is extracted into TBME (10 vol). Water (10 vol) is charged to the organic layer, and the product is extracted into the aqueous layer. The aqueous layer is washed with TBME (4×5 vol), then acidified with 5N HCl (3 vol) and extracted into TBME (2×5 vol). The combined organic extracts are washed with water (3×5 vol) and 15% brine solution (4 vol), then dried over magnesium sulphate (0.2 wt) and filtered, washing through with further TBME (2 vol). The volatiles are removed under vacuum to afford a tan solid of D1 (1.02 kg; 96% th.; 146% w/w).

### Description 1 (Alternative Method)

### 4-(octyloxy)-3-(trifluoromethyl)benzoic acid (D1)

1-octanol (1.52 vol, 2.0 eq) is charged to a solution of 1M NaHMDS in THF (13.5 vol, 2.8 eq) and warmed to 55-60° C. A solution of 4-fluoro-3-(trifluoromethyl)benzoic acid (1 wt., 1 eq) in THF (2 vol) is charged, washing through with further THF (1 vol). The reaction mixture is stirred at 55-60° C. until deemed complete by HPLC (ca. 24 h, <4% area 4-fluoro-3-(trifluoromethyl)benzoic acid remaining). The reaction is then solvent swapped into water (10 vol) by distillation (remove ca. 15 vol distillate) to afford a tan slurry which is extracted into TBME (10 vol). Water (10 vol) is charged to the organic layer, and the product is extracted into the aqueous layer. The aqueous layer is washed with TBME (4×5 vol), then acidified with 5N HCl (3 vol) and extracted into TBME (2×5 vol).* The combined organic extracts are washed with water (3×5 vol) and 15% brine solution (4 vol), then dried over magnesium sulphate (0.2 wt) and filtered, washing through with further TBME (2 vol). The volatiles are removed under vacuum to afford a tan solid of D1 (1.02 kg; 96% th.; 146% w/w). * alternatively, the product can be extracted into 2-Me THF (5 vol), washed with water (3×5 vol) and azeodried by put-and-take distillation with 2-Me THF (ca. 3×5 vol) to provide a dry 2-Me THF solution of D1 for use directly in the next stage.

### Description 2

### 4-(octyloxy)-3-(trifluoromethyl)benzohydrazide (D2)

D1 (1 wt., 1 eq) is dissolved in 2-Me THF (4 vol) and CDl (0.66 wt., 1.3 eq) is charged, rinsing in with 2-Me THF (1 vol). The mixture is stirred at 25±5° C. until activation is deemed complete by HPLC (ca. 15 min, ≦2% a/a D1 remaining). The solution is then added slowly to hydrazine hydrate (35% solution; 0.58 vol, 2 eq) in 2-Me THF (2 vol), rinsing in with further 2-MeTHF (1 vol). The resultant reaction mixture is stirred at 25±5° C. until reaction is deemed complete by HPLC (ca. 30 min, ≦2% a/a activated intermediate remaining). The reaction is washed successively with 1M HCl (2×5 vol) and 15% w/w aqueous potassium carbonate solution (4 vol), then dried over magnesium sulphate (0.5 wt) and filtered, washing through with further 2-Me THF (2 vol). The volatiles are removed under vacuum to afford a tan solid of D2 (1.07 kg; 101% th. corrected for residual solvent).

### Description 2 (Alternative Method)

### 4-(octyloxy)-3-(trifluoromethyl)benzohydrazide (D2)

D1 (1 wt., 1 eq) is dissolved in 2-Me THF (4 vol) and CDl (0.66 wt., 1.3 eq) is charged, rinsing in with 2-Me THF (1 vol). The mixture is stirred at 25±5° C. until activation is deemed complete by HPLC (ca. 15 min, ≦2% a/a D1 remaining). The solution is then added slowly to hydrazine hydrate (2 eq) in 2-Me THF (2 vol), rinsing in with further 2-MeTHF (1 vol). The resultant reaction mixture is stirred at 25±5° C. until reaction is deemed complete by HPLC (ca. 30 min, ≦2% a/a activated intermediate remaining). The reaction is washed successively with 1M HCl (2×5 vol) and 15% w/w aqueous potassium carbonate solution (4 vol), then dried over magnesium sulphate (0.5 wt) and filtered, washing through with further 2-Me THF (2 vol).* The volatiles are removed under vacuum to afford a tan solid of D2 (1.07 kg; 101% th. corrected for residual solvent). * alternatively, after the aqueous washes the product solution can be azeodried by put-and-take distillation with 2-Me THF (ca. 3×5 vol) to provide a dry 2-Me THF solution of D2 for use directly in the next stage.

### Description 3A and 3B

### 1,1-dimethylethyl (4S)-2,2,4-trimethyl-4-[(2-{[4-(octyloxy)-3-(trifluoromethyl)phenyl]carbonyl}hydrazino)carbonyl]-1,3-oxazolidine-3-carboxylate (D3A)

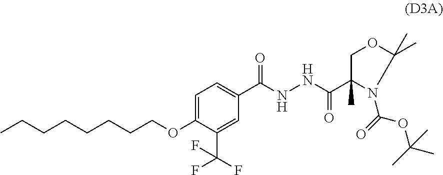

### 1,1-di methylethyl (2R,4S)-2-(1,1-dimethylethyl)-4-methyl-4-[(2-{[4-(octyloxy)-3-(trifluoromethyl)phenyl]carbonyl}hydrazino)carbonyl]-1,3-oxazolidine-3-carboxylate (D3B)

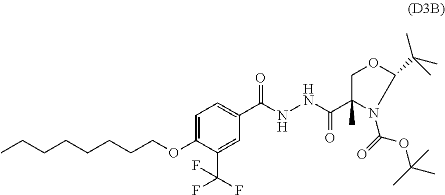

(4S)-3-{[(1,1-dimethylethyl)oxy]carbonyl}-2,2,4-trimethyl-1,3-oxazolidine-4-carboxylic acid (0.82 wt., 1.05 eq) or (2R,4S)-2-(1,1-dimethylethyl)-3-{[(1,1-dimethylethyl)oxy]carbonyl}-4-methyl-1,3-oxazolidine-4-carboxylic acid (0.91 wt., 1.05 eq) is dissolved in 2-Me THF (2.5 vol) and CDl (0.58 wt., 1.2 eq) is charged, washing in with 2-Me THF (0.5 vol). The reaction is heated at 40±5° C. and stirred at this temperature until deemed complete by HPLC (ca. 2 h). 2-propanol (0.2 vol) is charged and the reaction is stirred at 40±5° C. for ca. 30 minutes. A solution of 4-(octyloxy)-3-(trifluoromethyl)benzohydrazide (1 wt., 1 eq) in 2-Me THF (4.5 vol) is charged, washing in with 2-Me THF (0.5 vol), and the resulting solution is stirred for at least 3 hours at 40±5° C. until reaction deemed complete by HPLC. The mixture is cooled to 20±5° C., and washed successively with 1M HCl (2×3 vol), 5% w/w NaHCO₃ solution (4 vol) and water (2 vol). The organic layer is azeodried by put and take distillation of 2-Me THF (ca. 2×5 vol) and adjusted to 9 vol to provide a dry D3A or D3B solution in 2-MeTHF (˜20% w/w solution) for use directly in the next stage.

### Description 3A and 3B (Alternative Method)

### 1,1-dimethylethyl (4S)-2,2,4-trimethyl-4-[(2-{[4-(octyloxy)-3-(trifluoromethyl)phenyl]carbonyl}hydrazino)carbonyl]-1,3-oxazolidine-3-carboxylate (D3A)

### 1,1-dimethylethyl (2R,4S)-2-(1,1-dimethylethyl)-4-methyl-4-[(2-{[4-(octyloxy)-3-(trifluoromethyl)phenyl]carbonyl}hydrazino)carbonyl]-1,3-oxazolidine-3-carboxylate (D3B)

(4S)-3-{[(1,1-dimethylethyl)oxy]carbonyl}-2,2,4-trimethyl-1,3-oxazolidine-4-carboxylic acid (0.82 wt., 1.05 eq) or (2R,4S)-2-(1,1-dimethylethyl)-3-{[(1,1-dimethylethyl)oxy]carbonyl}-4-methyl-1,3-oxazolidine-4-carboxylic acid (0.91 wt., 1.05 eq) is dissolved in 2-Me THF and CDl (0.58 wt., 1.2 eq) is charged, washing in with 2-Me THF. The reaction is heated at 40±5° C. and stirred at this temperature until deemed complete by HPLC (ca. 2 h). 2-propanol (0.2 vol) is charged and the reaction is stirred at 40±5° C. for ca. 30 minutes. A solution of 4-(octyloxy)-3-(trifluoromethyl)benzohydrazide (1 wt., 1 eq) in 2-Me THF is charged, washing in with 2-Me THF, and the resulting solution is stirred for at least 3 hours at 40±5° C. until reaction deemed complete by HPLC. The mixture is cooled to 20±5° C., and washed successively with 1M HCl (2×3 vol), 5% w/w NaHCO₃ solution (4 vol) and water (2 vol). The organic layer is azeodried by put and take distillation of 2-Me THF (ca. 3×5 vol) and adjusted to ca. 9 vol to provide a dry D3A or D3B solution in 2-MeTHF (˜20% w/w solution) for use directly in the next stage.

### Description 4A and 4B

### 1,1-dimethylethyl (4S)-2,2,4-trimethyl-4-{5-[4-(octyloxy)-3-(trifluoromethyl)phenyl]-1,3,4-thiadiazol-2-yl}-1,3-oxazolidine-3-carboxylate (D4A)

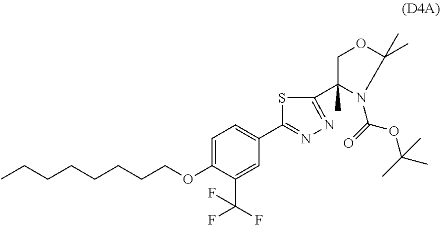

### 1,1-dimethylethyl (2R,4S)-2-(1,1-dimethylethyl)-4-methyl-4-{5-[4-(octyloxy)-3-(trifluoromethyl)phenyl]-1,3,4-thiadiazol-2-yl}-1,3-oxazolidine-3-carboxylate (D4B)

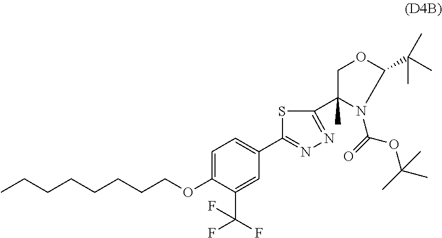

Lawesson's reagent (1.34 wt., 1.1 eq) is charged to the solution of D3A or D3B in 2-MeTHF, washing in with 2-Me THF (0.5 vol). The reaction is warmed to 70±5° C. and heated at this temperature for at least 1 h, until the reaction is deemed complete by HPLC (≦2% a/a residual D3A/D3B). The reaction is allowed to cool to 20±5° C. and is washed with 5N NaOH (2×2.5 vol), and then with 15% brine (2 vol). The organic phase is solvent swapped into isopropyl acetate by put and take distillation and adjusted to 10 vol to provide a D4A or D4B solution in isopropyl acetate (˜20% w/w solution).

### Description 4A and 4B (Alternative Method)

### 1,1-dimethylethyl (4S)-2,2,4-trimethyl-4-{5-[4-(octyloxy)-3-(trifluoromethyl)phenyl]-1,3,4-thiadiazol-2-yl}-1,3-oxazolidine-3-carboxylate (D4A)

### 1,1-dimethylethyl (2R,4S)-2-(1,1-dimethylethyl)-4-methyl-4-{5-[4-(octyloxy)-3-(trifluoromethyl)phenyl]-1,3,4-thiadiazol-2-yl}-1,3-oxazolidine-3-carboxylate (D4B)

Lawesson's reagent (1.34 wt., 1.1 eq) is charged to the solution of D3A or D3B in 2-MeTHF, washing in with 2-Me THF. The reaction is warmed to 70±5° C. and heated at this temperature for at least 1 h, until the reaction is deemed complete by HPLC (≦2% a/a residual D3A/D3B). The reaction is allowed to cool to 20±5° C. and is washed with 5N NaOH (2×2.5 vol), and then with 15% brine (2 vol). The organic phase is solvent swapped into isopropyl acetate by put and take distillation and adjusted to ca. 10 vol to provide a D4A or D4B solution in isopropyl acetate (˜20% w/w solution).

### Description 5

### (2S)-2-amino-2-{5-[4-(octyloxy)-3-(trifluoromethyl)phenyl]-1,3,4-thiadiazol-2-yl}-1-propanol (D5)

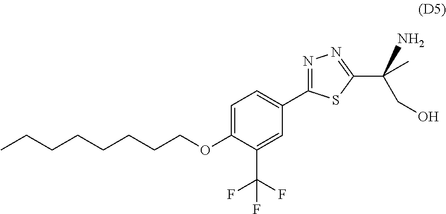

Water (1 vol) and conc. sulfuric acid (0.2 vol, 1.2 eq) are added to D4A or D4B in isopropyl acetate (˜20% w/w solution) and the reaction is warmed to 70±5° C., until deemed compete by HPLC (˜6 h). Water (5 vol) is added [for D4B input, trimethyl acetaldehyde is removed by distillation by put-take of isopropyl acetate (5 vol)] and the reaction is cooled to 20±5° C.]. The reaction is washed with 2N NaOH (5×3 vol), then 15% brine (3 vol). The solution is concentrated to 3 vol by distillation to provide an isopropyl acetate solution of D5 (˜35% w/w solution) for use directly in the next stage.

### Description 5 (Alternative Method)

### (2S)-2-amino-2-{5-[4-(octyloxy)-3-(trifluoromethyl)phenyl]-1,3,4-thiadiazol-2-yl}-1-propanol (D5)

Water (1 vol) and conc. sulfuric acid (0.2 vol, 1.2 eq) are added to D4A or D4B in isopropyl acetate (˜20% w/w solution) and the reaction is warmed to 70±5° C., until deemed compete by HPLC (˜6 h). Water (5 vol) is added [for D4B input, trimethyl acetaldehyde is removed by distillation by put-take of isopropyl acetate (5 vol)] and the reaction is cooled to 20±5° C. The reaction is washed with 2N NaOH (5×3 vol), then 15% brine (3 vol). The solution is adjusted to 9 vol by addition of isopropyl acetate to provide an isopropyl acetate solution of D5 (˜12% w/w solution) for use directly in the next stage.

### EXAMPLE 1

### (2S)-2-amino-2-{5-[4-(octyloxy)-3-(trifluoromethyl)phenyl]-1,3,4-thiadiazol-2-yl}-1-propanol hemi-fumarate (E1)

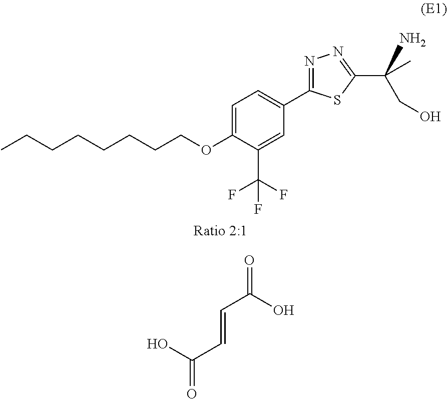

The isopropyl acetate solution of D5 is diluted with further isopropyl acetate and fumaric acid is charged. The reaction is warmed to provide a [hazy] solution, which is washed with water. The solution is azeodried by put-take distillation using further isopropyl acetate, and then clarified by filtration through a ≦5 micron filter, washing through with further isopropyl acetate. Crystallisation is established by cooling, and the resulting slurry is further cooled to 20±5° C. The solids are collected by filtration and washed with isopropyl acetate, then dried under vacuum to provide a white solid of E1.

In one particular batch, using the protocol outlined for Example 1, ca. 35% w/w solution of D5 in isopropyl acetate (2.89 kg solution) was diluted with isopropyl acetate (5.6 L) and treated with fumaric acid (0.14 kg). The mixture was heated to 70° C., washed with water (3×1.6 L) and the solution dried by put-take distillation with further isopropyl acetate (total 15.5 L). The solution was clarified through a 5 micron filter and the lines washed through with further isopropyl acetate (0.8 L). The solution was cooled to 50±5° C. at which point crystallisation initiated. The slurry was aged at 50±5° C. for 2.5 h then allowed to cool to 20±5° C. The solids were isolated by filtration, washed with isopropyl acetate (3×2 L) and dried under vacuum at 50±5° C. to provide E1 as a white solid (0.64 kg). Typical differential scanning calorimetry (DSC)/thermalgravimetric analysis (TGA) shows melt at ˜111-114° C.

### EXAMPLE 1

### Alternative Method

### (2S)-2-amino-2-{5-[4-(octyloxy)-3-(trifluoromethyl)phenyl]-1,3,4-thiadiazol-2-yl}-1-propanol hemi-fumarate (E1)

Fumaric acid is charged to the isopropyl acetate solution of D5 and the reaction is warmed to provide a [hazy] solution, which is washed with water. The solution is azeodried by put-take distillation using further isopropyl acetate, and then clarified by filtration through a ≦5 micron filter, washing through with further isopropyl acetate. The solution is concentrated to 10 vol by distillation, cooled to 60±3° C. and seeded with authentic GSK1842799C. The slurry is further cooled to 20±5° C. and the solids are collected by filtration and washed with isopropyl acetate, then dried under vacuum to provide a white solid of E1.

In one particular batch, using the protocol outlined for Example 1, ca. 12% w/w solution of D5 in isopropyl acetate (ca. 76 L solution) was treated with fumaric acid (1.44 kg). The mixture was heated to 70° C. to provide a hazy solution, then cooled to 62±2° C. and washed with water (2×17 L). The solution was dried by put-take distillation with further isopropyl acetate, clarified through a 5 micron filter and the lines washed through with further isopropyl acetate. The solution was concentrated to ca. 10 vol then cooled to 60±3° C. and seeded with GSK1842799C crystals (8.4 g). The resulting slurry was aged at 60±3° C. for 1 h then allowed to cool to 20±5° C. The solids were isolated by filtration, washed with isopropyl acetate (3×21 L) and dried under vacuum at 50±5° C. to provide E1 as a white solid (5.1 kg). Typical differential scanning calorimetry (DSC)/thermalgravimetric analysis (TGA) shows melt at ˜111-114° C. 1H NMR (400 MHz, DMSO-d₆) δ ppm 0.85 (3H, t, J=6.9 Hz) 1.20-1.37 (8H, m) 1.37-1.49 (2H, m) 1.47 (3H, s) 1.70-1.79 (2H, m) 3.52 (1H, d, J=10.5 Hz) 3.69 (1H, d, J=10.5 Hz) 4.19 (2H, t, J=6.2 Hz) 4.90-6.35 (3H, bs) 6.58 (1H, s) 7.41 (1H, d, J=8.8 Hz) 8.11 (1H, d, J=2.0 Hz) 8.15 (1H, dd, J=8.7, 2.1 Hz).

What is claimed is:

1. (2S)-2-amino-2-{5-[4-(octyloxy)-3-(trifluoromethyl)phenyl]-1,3,4-thiadiazol-2-yl}-1-propanol hemi-fumarate.

2. A method of treating a condition or disorder mediated by a S1P1 receptor in a mammal in need thereof, comprising: administering a therapeutically effective amount of a compound according to claim 1.

3. A method according to claim 2, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin or non-insulin dependant diabetes.

4. A method according to claim 3, wherein the condition is multiple sclerosis.

5-7. (canceled)

8. A pharmaceutical composition comprising the compound according to claim 1.

9. A method of treating a condition or disorder mediated by a S1P1 receptor in a mammal, including a human, in need thereof, comprising: administering to the mammal a therapeutically effective amount of (2S)-2-amino-2-{5-[4-(octyloxy)-3-(trifluoromethyl)phenyl]-1,3,4-thiadiazol-2-yl}-1-propanol or a pharmaceutically acceptable salt thereof.

10. A method of treatment according to claim 9, wherein the condition is multiple sclerosis.

11. A method according to claim 9, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin or non-insulin dependant diabetes.
